(19) United States
(12) Patent Application Publication
(10) Pub. No.: US 2021/0252183 A1
(43) Pub. Date: Aug. 19, 2021

(54) MEDICAL COMPOSITE MATERIAL AND PREPARATION METHOD THEREOF

(71) Applicant: Shandong Juntai Medical Protective Equipment Technology Co., Ltd., Jinan (CN)

(72) Inventor: Jiehu XING, Jinan (CN)

(21) Appl. No.: 17/306,883

(22) Filed: May 3, 2021

Related U.S. Application Data
(63) Continuation-in-part of application No. PCT/CN2020/079456, filed on Mar. 16, 2020.

(30) Foreign Application Priority Data
Dec. 18, 2019 (CN) 201911305690.0

(51) Int. Cl. A61L 15/46; A61L 15/22; B32B 5/02; B32B 7/12; B32B 27/36; B32B 27/40; B32B 27/12

(52) U.S. Cl. CPC A61L 15/46; A61L 15/225; B32B 5/024; B32B 7/12; B32B 27/36; B32B 2307/724; B32B 27/12; A61L 2300/404; B32B 2262/0284; B32B 2307/726; B32B 27/40

(57) ABSTRACT

The disclosure provides a medical composite material including 55-65 parts by weight of a polyethylene terephthalate (PET) fiber, 8-12 parts by weight of a carbon ion filament fiber, 18-23 parts by weight of a polyurethane (PU) film, and 5-15 parts by weight of an excipient.

### CROSS-REFERENCE TO RELAYED APPLICATIONS

This application is a continuation-in-part of International Patent Application No. PCT/CN2020/079456 with an international filing date of Mar. 16, 2020, designating the United States, now pending, and further claims foreign priority benefits to Chinese Patent Application No. 201911305690.0 filed Dec. 18, 2019. The contents of all of the aforementioned applications, including any intervening amendments thereto, are incorporated herein by reference. Inquiries from the public to applicants or assignees concerning this document or the related applications should be directed to: Matthias Scholl P.C., Attn.: Dr. Matthias Scholl Esq., 245 First Street, 18th Floor, Cambridge, Mass. 02142.

### BACKGROUND

The disclosure relates to a medical composite material and preparation method thereof.

The commonly used materials of medical fabrics are cotton and disposable non-woven materials.

Cotton material is permeable to blood, body fluid, flushing fluid and other pollutants, and is not antibacterial. This leads to infection risks of the medical staff and patients.

The disposable non-woven materials have a limited water vapor transmission rate (WVTR), leading to bad user experience. The use of the disposable non-woven materials is costly, and the treatment thereof tends to produce dioxin, which is environmentally unfriendly.

### SUMMARY

The disclosure provides a medical composite material, comprising 55-65 parts by weight of a polyethylene terephthalate (PET) fiber, 8-12 parts by weight of a carbon ion filament fiber, 18-23 parts by weight of a polyurethane (PU) film, and 5-15 parts by weight of an excipient.

In a class of this embodiment, the medical composite material comprises 60 parts by weight of the polyethylene terephthalate fiber, 10 parts by weight of the carbon ion filament fiber, 20 parts by weight of the polyurethane film, and 10 parts by weight of the excipient; the excipient is a polyurethane adhesive.

The disclosure also provides a method for preparing the medical composite material, the method comprising:
preparing a first polyester filament fiber, deforming the first polyester filament fiber in air to reduce a hardness and smoothness thereof, where the first polyester filament fiber is polyethylene terephthalate fiber;
permeating carbon ions into a second polyester filament fiber, where the second polyester filament fiber has a circular cross section; coating the second polyester filament fiber with the carbon ions, thereby yield a conductive fiber with a thickness of 1 micrometer;
weaving the deformed first polyester filament fiber and the conductive fiber into an antistatic textile material, where a distance between two adjacent conductive fibers is 1 cm;
shaping polyurethane into an antibacterial thin film having a thickness of 0.012-0.035 mm, where the antimicrobial thin film has 1.395 billion micropores per square centimeter, each micropore is 280 nm in diameter; and
disposing the antibacterial thin film between the deformed first polyester filament fiber and the conductive fiber, compounding the deformed first polyester filament fiber, the antibacterial thin film, and the conductive fiber in the presence of a polyurethane adhesive by roll compounding process, to yield the medical composite material.

The method further comprises printing and dyeing the medical composite material, which comprises:
printing and dyeing a sample of the medical composite material, where the sample is 40 cm x100 cm in size, immersing the sample in a printing and dying additive in a glass or enamel container, and immersing the sample in a hypochlorite solution for 60 min, where an effective chlorine content in the hypochlorite solution is 2 g/L, and a pH of the hypochlorite solution is 11.0±0.2; and
examining the sample, if it is qualified, printing and dyeing the medical composite material in batches.

The following advantages are associated with the medical composite material of the disclosure.

The medical composite material of the disclosure is waterproof, impermeable and resistant to bacteria, and prevents the penetration of blood, body fluid, flushing fluid and pollutants, thus completely eliminating the infection of patients and protecting the medical staff and patients. The medical composite material is permeable to moisture, which increases the comfort of the medical staff and patients and is conducive to the use of surgical clothing, surgical sheet and instrument sheet. The medical composite material is antistatic, avoiding the operation risk caused by static electricity. No floccule is produced when in use, avoiding the infection risk. The medical composite material is resistant to hypochlorite rinsing, increases washing times and reduces single use cost. In use, no dioxin and other carcinogens is produced. In addition to the medical field, the medical composite material can also be used for the aged, infant and military fields.

### DETAILED DESCRIPTION

To further illustrate, embodiments detailing a medical composite material are described below. It should be noted that the following embodiments are intended to describe and not to limit the disclosure.

### Example 1

Provided is a medical composite material comprising 60 parts by weight of the polyethylene terephthalate fiber, 10 parts by weight of the carbon ion filament fiber, 20 parts by weight of the polyurethane film, and 10 parts by weight of the excipient. Specifically, the excipient is a polyurethane adhesive.

The medical composite material is prepared as follows:

(1) preparing a first polyester filament fiber, deforming the first polyester filament fiber in air to reduce the hardness and smoothness thereof; the first polyester filament fiber is polyethylene terephthalate fiber;

(2) permeating carbon ions into a second polyester filament fiber, where the second polyester filament fiber has a circular cross section; coating the second polyester filament fiber with the carbon ions, thereby yield a conductive fiber with a thickness of 1 micrometer;

(3) weaving the deformed first polyester filament fiber and the conductive fiber into an antistatic textile material, where a distance between two adjacent conductive fibers is 1 cm;

(4) shaping polyurethane into an antibacterial thin film having a thickness of 0.015-0.025 mm, where the antimicrobial thin film has 1.395 billion micropores per square centimeter, each micropore is 280 nm in diameter; and

(5) disposing the antibacterial thin film between the deformed first polyester filament fiber and the conductive fiber, compounding the deformed first polyester filament fiber, the antibacterial thin film, and the conductive fiber in the presence of a polyurethane adhesive by roll compounding process, to yield the medical composite material.

### Example 2

Provided is a medical composite material comprising 63 parts by weight of the polyethylene terephthalate fiber, 12 parts by weight of the carbon ion filament fiber, 22 parts by weight of the polyurethane film, and 13 parts by weight of the excipient. Specifically, the excipient is a polyurethane adhesive.

The medical composite material is prepared as follows:

(1) preparing a first polyester filament fiber, deforming the first polyester filament fiber in air to reduce the hardness and smoothness thereof; the first polyester filament fiber is polyethylene terephthalate fiber;

(2) permeating carbon ions into a second polyester filament fiber, where the second polyester filament fiber has a circular cross section; coating the second polyester filament fiber with the carbon ions, thereby yield a conductive fiber with a thickness of 1 micrometer;

(3) weaving the deformed first polyester filament fiber and the conductive fiber into an antistatic textile material, where a distance between two adjacent conductive fibers is 1 cm;

(4) shaping polyurethane into an antibacterial thin film having a thickness of 0.012-0.035 mm, where the antimicrobial thin film has 1.395 billion micropores per square centimeter, each micropore is 280 nm in diameter; and

(5) disposing the antibacterial thin film between the deformed first polyester filament fiber and the conductive fiber, compounding the deformed first polyester filament fiber, the antibacterial thin film, and the conductive fiber in the presence of a polyurethane adhesive by roll compounding process, to yield the medical composite material.

### Example 3

Provided is a medical composite material comprising 56 parts by weight of the polyethylene terephthalate fiber, 9 parts by weight of the carbon ion filament fiber, 18 parts by weight of the polyurethane film, and 8 parts by weight of the excipient. Specifically, the excipient is a polyurethane adhesive.

The medical composite material is prepared as follows:

(1) preparing a first polyester filament fiber, deforming the first polyester filament fiber in air to reduce the hardness and smoothness thereof; the first polyester filament fiber is polyethylene terephthalate fiber;

(2) permeating carbon ions into a second polyester filament fiber, where the second polyester filament fiber has a circular cross section; coating the second polyester filament fiber with the carbon ions, thereby yield a conductive fiber with a thickness of 1 micrometer;

(3) weaving the deformed first polyester filament fiber and the conductive fiber into an antistatic textile material, where a distance between two adjacent conductive fibers is 1 cm;

(4) shaping polyurethane into an antibacterial thin film having a thickness of 0.025-0.035 mm, where the antimicrobial thin film has 1.395 billion micropores per square centimeter, each micropore is 280 nm in diameter; and

(5) disposing the antibacterial thin film between the deformed first polyester filament fiber and the conductive fiber, compounding the deformed first polyester filament fiber, the antibacterial thin film, and the conductive fiber in the presence of a polyurethane adhesive by roll compounding process, to yield the medical composite material.

### Example 4

In certain embodiments, the method further comprises printing and dyeing the prepared medical composite material to meet the standard Grade Four of GB/T7069-1997, which comprises:

1) printing and dyeing of a sample: printing and dyeing a sample of the medical composite material, where the sample is 40 cm x100 cm in size, immersing the sample in a printing and dying additive in a glass or enamel container, and immersing the sample in a hypochlorite solution for 60 min, where the effective chlorine content in the hypochlorite solution is 2 g/L, and a pH of the hypochlorite solution is 11.0±0.2; and

2) printing and dyeing in batches: examining the sample, if it is qualified, printing and dyeing the medical composite material in batches.

Table 1 shows the test result of printing and dyeing of the sample of the medical composite material:

TABLE 1

| Characteristic | Actual test result | Test methods |
|---|---|---|
| Charge, unit: uC/piece | 0.56 | GB12014-2009 |
| | | 22° C., 34% RH |
| Water resistance (hydrostatic | >3000 | GB/T 4744-2013 |
| pressure method) (mm H₂O) | | (60 cm H²O/min, water |
| (the observe side) | | temperature of 20° C.) |
| Colorfastness (grade) to | 4 | GB/T 7069-1997 |
| hypochlorite bleaching, | | |
| discoloration | | |
| Water vapor permeability | 7.8 × 10³ | GB/T 12704.2-2009 f |
| (g/(m² · d)) (the | | Method B (at a |
| obverse side towards | | temperature of 38° C. |
| the water) | | and a relative |
| | | humidity of 50%) |
| Moisture permeability | 9774.9 | GB/T 12704.2-2009 |
| (g/m²/24 hrs) after | | Method B; inverted |
| washing 20 times, at a | | cup evaporation |
| temperature of | | method |
| 38° C. ± 2° C. and a | | |
| humidity of 50% ± 2% | | |
| Hydrostatic pressure after | 8760 | GB/T 4744-2013; |
| washing 20 times (mm) | | water pressure rise |
| | | rate: 10 cm H₂O/min |
| Moisture permeability | 10586.0 | GB/T 12704.2-2009 |
| (g/m²/24 hrs) after | | Method B; inverted |
| washing 100 times, at a | | cup evaporation |
| temperature of 38° C. ± | | method |
| 2° C. and a humidity of | | |
| 50% ± 2% | | |
| Hydrostatic pressure after | 855 | GB/T 4744-2013; |
| washing 100 times (mm) | | water pressure rise |
| | | rate: 10 cm H₂O/min |

The test result showed that, when cotton cloth was used as a control sample, the cotton cloth was penetrated by Staphylococcus aureus under wet conditions. The total number of colonies growing on 5 medium plates was more than 5. Under dry conditions, the total number of colonies penetrated in 10 samples was 187 colony-forming unit (CFU), which was much higher than 15 CFU. The medical composite material of the disclosure can effectively prevent the penetration of Staphylococcus aureus under wet conditions and black spore variants of Bacillus subtilis under dry conditions. The detection result showed, the cleanliness of the medical composite material of the disclosure—microorganism ≤300 CFU/dm²; cleanliness—particulate matters ≤3.5 index of particulate matters (IPM); flocculus ≤4.0 log 10; dry and wet bursting strength ≥40 kpa; dry and wet fracture strength: ≥20 N.

It will be obvious to those skilled in the art that changes and modifications may be made, and therefore, the aim in the appended claims is to cover all such changes and modifications.

What is claimed is:

1. A medical composite material, comprising 55-65 parts by weight of a polyethylene terephthalate (PET) fiber, 8-12 parts by weight of a carbon ion filament fiber, 18-23 parts by weight of a polyurethane (PU) film, and 5-15 parts by weight of an excipient.

2. The medical composite material of claim 1, comprising 60 parts by weight of the polyethylene terephthalate fiber, 10 parts by weight of the carbon ion filament fiber, 20 parts by weight of the polyurethane film, and 10 parts by weight of the excipient; wherein the excipient is a polyurethane adhesive.

3. A method for preparing the medical composite material of claim 1, the method comprising:
preparing a first polyester filament fiber, deforming the first polyester filament fiber in air to reduce a hardness and smoothness thereof, wherein the first polyester filament fiber is the polyethylene terephthalate fiber;
permeating carbon ions into a second polyester filament fiber, wherein the second polyester filament fiber has a circular cross section; coating the second polyester filament fiber with the carbon ions, thereby yield a conductive fiber with a thickness of 1 micrometer;
weaving the first polyester filament fiber deformed and the conductive fiber into an antistatic textile material, wherein a distance between two adjacent conductive fibers is 1 cm;
shaping polyurethane into an antibacterial thin film having a thickness of 0.012-0.035 mm, wherein the antimicrobial thin film has 1.395 billion micropores per square centimeter, each micropore is 280 nm in diameter; and
disposing the antibacterial thin film between the deformed first polyester filament fiber and the conductive fiber, compounding the deformed first polyester filament fiber, the antibacterial thin film, and the conductive fiber in the presence of a polyurethane adhesive by roll compounding process, to yield the medical composite material.

4. The method of claim 3, further comprising printing and dyeing the medical composite material, which comprises:
printing and dyeing a sample of the medical composite material, wherein the sample is 40 cm×100 cm in size; immersing the sample in a printing and dying additive in a glass or enamel container, and immersing the sample in a hypochlorite solution for 60 min, wherein an effective chlorine content in the hypochlorite solution is 2 g/L, and a pH of the hypochlorite solution is 11.0±0.2; and
examining the sample, if it is qualified, printing and dyeing the medical composite material in batches.
